(19) United States
(12) Patent Application Publication
(10) Pub. No.: US 2017/0095497 A1
(43) Pub. Date: Apr. 6, 2017

(54) TRANSIENT INHIBITION OF ADENOSINE KINASE AS AN ANTI-EPILEPTOGENESIS TREATMENT

(71) Applicant: Legacy Emanuel Hospital & Health Center, Portland, OR (US)

(72) Inventors: Detlev Boison, Portland, OR (US); Ursula Susan Sandau, Beaverton, OR (US)

(73) Assignee: Legacy Emanuel Hospital & Health Center, Portland, OR (US)

(21) Appl. No.: 15/284,432

(22) Filed: Oct. 3, 2016

Related U.S. Application Data
(60) Provisional application No. 62/236,091, filed on Oct. 1, 2015.

(51) Int. Cl. A61K 31/7064; A61K 9/00

(52) U.S. Cl. CPC A61K 31/7064; A61K 9/0053

(57) ABSTRACT

Methods of anti-epileptogenesis treatment in which adenosine kinase (ADK) activity or expression is inhibited only transiently to provide a long-term benefit to a non-epileptic or epileptic subject. In an exemplary method, a therapeutically effective amount of an ADK inhibitor may be administered to a human non-epileptic subject over a finite, predetermined treatment period having a duration of less than two months. The non-epileptic subject may have sustained a precipitating event with a known risk to trigger latent development of an acquired form of epilepsy. Administration of the ADK inhibitor to the subject may be stopped at the end of the treatment period for at least the longer of (i) six months and (ii) ten times the duration of the treatment period. The step of administering may reduce the chance of the subject having seizures caused by the acquired form of epilepsy for an extended period following the end of the treatment period.

### CROSS-REFERENCE TO PRIORITY APPLICATION

This application is based upon and claims the benefit under 35 U.S.C. §119(e) of U.S. Provisional Patent Application Ser. No. 62/236,091, filed Oct. 1, 2015, which is incorporated herein by reference in its entirety for all purposes.

### INTRODUCTION

Epilepsy, such as temporal lobe epilepsy, presents as an incapacitating neurological syndrome comprised of recurrent, unprovoked seizures and associated comorbidities. Increasing the level of adenosine in the brain has been proposed to treat the symptoms of temporal lobe epilepsy. For example, the level of adenosine can be increased with an adenosine kinase (ADK) inhibitor, to slow the conversion of adenosine to adenosine monophosphate (AMP) by ADK.

ADK inhibitors were in pre-clinical drug development in the past, with a peak of drug-development activity between 2000 and 2005. Those studies were aimed at using ADK inhibitors long-term for symptomatic treatment of chronic disorders associated with reduced adenosine levels, namely, epilepsy, chronic pain, and persistent inflammation. However, the long-term use of ADK inhibitors was found to be unacceptably toxic (e.g., for the liver) and to produce debilitating side effects including strong sedation. Therefore, around 2005, drug development efforts with ADK inhibitors were halted.

### SUMMARY

The present disclosure provides methods of anti-epileptogenesis treatment in which adenosine kinase (ADK) activity or expression is inhibited only transiently to provide a long-term benefit to a non-epileptic or epileptic subject. In an exemplary method, a therapeutically effective amount of an inhibitor of ADK activity or expression may be administered to a human non-epileptic subject over a finite, predetermined treatment period having a duration of less than two months. The non-epileptic subject may have sustained a precipitating event with a known risk to trigger latent development of an acquired form of epilepsy. Administration of the inhibitor to the subject may be stopped at the end of the treatment period for at least the longer of (i) six months and (ii) ten times the duration of the treatment period. The step of administering may reduce the chance of the subject having seizures caused by the acquired form of epilepsy for an extended period following the end of the treatment period.

### BRIEF DESCRIPTION OF THE DRAWINGS

FIG. 1 is a set of images of mouse hippocampal sections exposed to a Nissl stain, an ADK antibody, or a 5-methylcytosine antibody, with the sections representing hippocampi at time zero (control), 3 days (3 d), or 7 days (7 d) after intrahippocampal administration of kainic acid (KA) to produce status epilepticus (SE). At 7 d increases in both ADK and 5 mC become evident in the KA-injected subjects.

FIG. 2 is a flowchart illustrating how an increase in adenosine kinase activity during the latent phase of epileptogenesis may increase DNA methyltransferase (DNMT) activity, resulting in more cytosine methylation of hippocampal DNA.

FIGS. 3 and 4 are graphs showing the level of DNMT activity present one hour after administration of vehicle alone or various inhibitors affecting the level of adenosine (FIG. 3) or directly inhibiting DNMT (FIG. 4).

FIG. 5 is a timeline showing the protocol used with murine subjects to induce epileptogenesis, administer an ADK inhibitor during a short-term treatment period, and record electrical activity within a long-term benefit period after the end of the treatment period.

FIGS. 6 and 7 are a pair of graphs respectively showing the frequency of seizures and the time spent in seizures at six weeks after triggering epileptogenesis, for control mice (no ADK inhibitor) and mice treated with an ADK inhibitor (ITU) once or twice daily according to the protocol of FIG. 5.

FIG. 8 is a pair of graphs respectively showing the frequency of seizures and the time spent in seizures at six weeks after triggering epileptogenesis, for control mice (no ADK inhibitor) and mice treated with an ADK inhibitor (ITU) twice daily according to the protocol of FIG. 5, where the data plotted in FIG. 8 were obtained in a partial repeat of the experiment of FIGS. 6 and 7.

FIGS. 9 and 10 are representative electroencephalogram (EEG) recordings taken at the six-week time point of the protocol of FIG. 5 from a control subject (FIG. 9) and an ITU-treated subject (FIG. 10).

FIG. 11 is a series of representative images from a histological analysis of hippocampal tissue sections obtained from mice at nine weeks according to the protocol of FIG. 5, with no KA and no ITU treatment as controls.

FIGS. 12-14 are a series of graphs plotting data from the histological analysis represented by FIG. 11.

### DETAILED DESCRIPTION

The present disclosure provides methods of anti-epileptogenesis treatment in which adenosine kinase (ADK) activity or expression is inhibited only transiently to provide a long-term benefit to a non-epileptic or epileptic subject. In an exemplary method, a therapeutically effective amount of an inhibitor of ADK activity or expression may be administered to a human non-epileptic subject over a finite, predetermined treatment period having a duration of less than two months. The non-epileptic subject may have sustained a precipitating event with a known risk to trigger latent development of an acquired form of epilepsy. Administration of the inhibitor to the subject may be stopped at the end of the treatment period for at least the longer of (i) six months and (ii) ten times the duration of the treatment period. The step of administering may reduce the chance of the subject having seizures caused by the acquired form of epilepsy for an extended period following the end of the treatment period.

Epileptogenesis is the development and progression of epilepsy. This process constitutes molecular and physiological changes that make a brain more susceptible to seizures. Epileptogenesis can be triggered by a precipitating event in the brain (e.g., traumatic injury, stroke, infection, fever, or the like). After the precipitating event, a “latent period” of weeks, months, or years follows, and then epileptic seizures begin. Once epilepsy is manifest, epileptogenesis is a continuing process that leads to gradual worsening of the disease (“seizures beget seizures”). This gradual worsening is termed progression. An anti-epileptogenic treatment can be initiated during the latent period to statistically reduce the chance of developing epilepsy, or after the onset of epilepsy to prevent or impede worsening of the disease, pharmacoresistance, and/or cognitive or psychiatric comorbidities.

The methods of the present disclosure rely on a short-term administration of an ADK inhibitor to provide a long lasting benefit to a subject. By restricting administration to a relatively narrow window of time, the subject can receive larger and/or more frequent doses of the ADK inhibitor than would be acceptable or even considered with long-term administration. Short-term administration also addresses concerns about toxicity and side effect of the ADK inhibitor, by minimizing the time of exposure to the inhibitor. Surprisingly, administering the ADK inhibitor well before symptoms of epilepsy appear is still effective.

The present disclosure demonstrates that the transient use of an exemplary ADK inhibitor can have lasting epilepsy-preventing effects. Using a mouse model of epileptogenesis, the present disclosure shows that a five-day treatment with an ADK inhibitor, administered twice daily early in the latent phase of epileptogenesis, prevents the later development of epilepsy. Accordingly, transient administration of an inhibitor of ADK activity or expression can have robust and lasting therapeutic effects. A transient treatment regimen with the inhibitor avoids the risks of chronic toxicities (e.g., to the liver), and the short-term side effects (e.g., sedation) associated with this transient regimen are clinically acceptable. Key findings presented here include (a) definition of a time window of therapeutic efficacy for mice (i.e., five days of treatment starting three days after the epileptogenesis trigger, and (b) dosing information (i.e., twice daily administration as opposed to once daily administration of a higher dose may be needed for the therapeutic effects).

Further aspects of the present disclosure are described in the following sections: (I) subjects, (II) inhibitors of ADK activity or expression, (III) short-term administration of inhibitors of ADK activity or expression, (IV) long-term benefits of treatment, and (V) examples.

### I. Subjects

A subject may receive an anti-epileptogenesis treatment as disclosed herein. The subject may be selected from any suitable animal species, but is typically human. In various embodiments, the subject may not or may be epileptic.

The subject may be a non-epileptic subject in need of treatment to discourage development of epilepsy. A non-epileptic subject, as used herein, is any subject who has not experienced recurrent, unprovoked seizures in the preceding two years and/or has never been diagnosed with epilepsy.

The non-epileptic subject may have sustained a precipitating event with a known risk to trigger latent development of an acquired form of epilepsy in non-epileptic subjects. The precipitating event can stress and/or injure the brain of the subject. The event itself may have a short duration, such as a duration of less than about 2 or 1 week(s); less than about 4, 2, or 1 day(s); or less than about 5 or 1 hour(s); among others. Alternatively, or in addition, the precipitating event may have occurred less than about one year; less than about 6, 4, 3, 2, or 1 month(s); less than about 2 or 1 week(s); less than about 5, 4, 3, 2 or 1 day(s); or less than about 6 or 2 hours before the non-epileptic subject begins receiving an inhibitor of ADK activity or expression. Exemplary precipitating events that can trigger development of an acquired form of epilepsy may include traumatic brain injury, hemorrhagic stroke, ischemic stroke, infection of the brain, febrile seizure, and status epilepticus. (Status epilepticus is an emergency condition in which the subject has a single seizure lasting longer than five minutes, or has a series of seizures that occur in rapid succession, without the subject regaining consciousness.) Acquired forms of epilepsy include any form of epilepsy not present at birth and associated with increased levels of adenosine kinase in a region of the brain (e.g., temporal lobe epilepsy). During latent development, symptoms of epilepsy, such as recurrent seizures, are absent. The latent development phase may last any suitable length of time, such as weeks, months, or years, before symptoms of epilepsy appear. Accordingly, the non-epileptic subject can experience a long-term benefit from treatment that reduces the risk of becoming epileptic for the length of an extended development phase, such as at least about six months, one year, or two years after receiving an inhibitor of ADK activity or expression.

In other embodiments, the subject may be an epileptic subject in need of treatment to discourage progression of epilepsy to a more severe form. (Once the epilepsy becomes more advanced, the inhibitor of ADK activity or expression may become much less effective for transient administration.) The epileptic subject may have a (currently) mild form of epilepsy and/or may have been diagnosed with epilepsy recently, where the epilepsy is a type associated with a reduced level of adenosine in the brain. Criteria for determining whether the epileptic subject is a candidate for treatment may include the frequency or total number of seizures and/or the duration/severity thereof, during a given time period, such as the preceding year, month, week, or the like. More particularly, a value for the frequency/number of seizures and/or seizure duration/severity may be compared with a threshold value, to determine whether a given epileptic subject has a sufficiently mild form of epilepsy to qualify for treatment. Alternatively, or in addition, the suitability of the epileptic subject for treatment may be determined based on how long the subject has had epilepsy. For example, the epileptic subject may qualify for treatment only if the subject received a first diagnosis of epilepsy within the preceding year or within the preceding 6, 4, 3, 2, or 1 month(s) from when administration of an inhibitor of ADK activity or expression would begin.

### II. Inhibitors of ADK Activity or Expression

The methods disclosed herein are performed with an inhibitor of ADK activity or expression. The inhibitor can be an “ADK inhibitor” capable of specifically reducing ADK enzyme activity, generally by interacting with ADK protein, or can be an “ADK expression inhibitor” capable of specifically reducing ADK expression. The unmodified term “inhibitor” is used herein to encompass both types of inhibitor. The inhibitor may be a single compound or may include two or more compounds. If the inhibitor includes two or more compounds, at least a subset of the compounds may be present together in the same pharmaceutical preparation or may be present in separate preparations, which may be administered separately to the subject.

Each ADK inhibitor may have any suitable properties. The ADK inhibitor may be a small molecule having a molecular weight of less than about 10, 5, or 2 kilodaltons, among others. In exemplary embodiments, the ADK inhibitor does not include DNA, RNA, or a nucleic acid analog, and/or does not contain a chain of five or more nucleotides. The ADK inhibitor may have a half maximal inhibitory concentration (IC50) of less than about 100 nM or 10 nM, among others, for inhibition of ADK activity. ADK inhibitors include nucleoside inhibitors and non-nucleoside inhibitors. In some embodiments, the adenosine kinase inhibitor is selected from the group consisting of adenosine analogs, pyridopyrimidine derivatives, and alkynylpyrimidine derivatives.

Exemplary nucleoside inhibitors (e.g., adenosine analogs) that may be suitable as ADK inhibitors include any of the following: 5-iodotubercidin (ITU) (4-amino-5-iodo-7 pyrrolo[2,3-d]pyrimidine); 5′-deoxy,5-iodotubercidin; 5′-amino-5′-deoxyadenosine; GP-3269 (7-(5-deoxy-β-D-ribofuranosyl)-N-(4-fluorophenyl)-5-phenyl-7H-pyrrolo[2,3-d]pyrimidin-4-amine); A-134974 (N7-[(1′R,2′S,3′R,4′S)-2′,3′-dihydroxy-4′-aminocyclopentyl]-4-amino-5-iodopyrrolopyrimidine); A-286501 (N7-((1′R,2′S,3′R,4′S)-2′,3′-dihydroxy-4′-amino-cyclopentyl)-4-amino-5-bromo-pyrrolo[2,3-c]pyrimidine; AraA (9-β-D-ribofuranosyladenine); GP-515 (4-amino-1-(5-am ino-5-deoxy-1-β-d-ribofuranosyl)-3-bromo-pyrazol[3,4-d] pyrimidine); GP-3269 (7-(5-deoxy-β-D-ribofuranosyl)-N-(4-fluorophenyl)-5-phenyl-7H-pyrrolo[2,3-d]pyrim idin-4-amine; and GP-3966 (4-N-(4-fluorophenyl)amino-5-phenyl-7-(β-D-erythrofuranosyl) pyrrolo[2,3-d]pyrimidine).

Exemplary non-nucleoside inhibitors that may be suitable include ABT-702 (4-amino-5-(3-bromophenyl)-7-(6-morpholino-pyridin-3-Apyrido[2,3-d]pyrim idine); 2-N, N-dimethyl-7-benzyl bispyrrolidino axazolo-pyrimidine; and the like.

In other embodiments, the inhibitor may be an inhibitor of ADK expression. Exemplary inhibitors of ADK expression include nucleic acids or analogs thereof. The inhibitor of ADK expression may include anti-ADK interfering RNA that binds specifically to an ADK gene and/or ADK RNA.

### III. Short-Term Administration of Inhibitors of ADK Activity or Expression

A therapeutically effective amount of an inhibitor of ADK expression or activity, in a pharmaceutically acceptable preparation, may be administered to the subject only transiently. Transient administration may be restricted to a finite, predetermined treatment period, and may constitute a predefined dose regimen of one or more doses of the inhibitor. The treatment period has a duration measured from the beginning of the first (or only) dose to the end of the last (or only) dose, with the duration expressed as an integer number of days after rounding up to the nearest whole day. (In other words, a treatment period beginning and ending in less than 24 hours has a duration of one day.) A suitable duration for the treatment period may be less than about 2 or 1 month(s); less than about 3, 2, or 1 week(s); or less than about 6, 5, 4, 3, or 2 days; among others. Accordingly, the treatment period may be only one day and/or a single dose. In some embodiments, the duration of the treatment period may be at least about 2, 3, 4, or 5 days; or at least about 1, 2, 3, or 4 weeks.

Any suitable dose regimen may be followed over the treatment period. The inhibitor may be administered in any suitable number of doses per day, such as 1, 2, 3, 4, 5, or more doses, or may be administered less than once per day, such as every other day, every third day, or the like. Each dose may be delivered over any suitable time period, which generally may be determined at least in part on the route of administration. For example, the dose may be administered relatively rapidly with a syringe or orally (such as in less than about one minute), or more slowly and continuously with a pump (such as over at least 10, 30, or 60 minutes, or even over more than one day).

The dose regimen may produce a concentration of inhibitor in the bloodstream and/or within the brain of the subject that is above the IC50 for any suitable fraction of the treatment period. (For an ADK (activity) inhibitor, the IC50 may be defined with respect to the nuclear or cytoplasmic form of ADK.) In some embodiments, the concentration may be above the IC50 each day of the treatment period and/or a majority (e.g., more than 50%, 60%, 75%, 80%, or 90%) of each day.

Administration of the inhibitor may be by any suitable route, such as oral, intravenous, intranasal, buccal, rectal, cutaneous, intradermal, intraperitoneal, directly to the brain, or the like. In some embodiments, the inhibitor may be administered substantially continuously, such as via an intravenous line, a patch, or an implanted pump, among others. In some embodiments, the administration may be systemic or preferentially to the brain. The route of administration may determine who administers the inhibitor to the subject. To exemplify, the inhibitor may be administered by a certified medical practitioner (e.g., an injection performed by a nurse or doctor at a medical facility) or may be self-administered by the subject (e.g., a pill taken orally at home).

Administration of the inhibitor is terminated at the end of the treatment period. The administration may be stopped for a stoppage period of at least about 6 or 9 months, or at least about 1 or 2 years, among others. Alternatively, or in addition, the administration may be stopped for a stoppage period of at least about 5, 10, 15, 20, 25, or 50 times the duration of the treatment period. Furthermore, administration of the inhibitor may be terminated indefinitely, or may be resumed after the end of the stoppage period. If resumed, the inhibitor may be administered again to the subject after the stoppage period for at least one further treatment period followed by another stoppage period. For example, the subject may be treated periodically (such as one short-term treatment per year) to discourage epileptogenesis.

In some embodiments, the subject may be treated with the inhibitor and caffeine (or theophylline) in a combination therapy during the treatment period. Each dose of the inhibitor and each dose of caffeine/theophylline may be administered together or separately. Caffeine may block the adenosine receptor mediated side effects (e.g., sedation) of the inhibitor while not influencing the epigenetic effect of the inhibitor, which may be adenosine-receptor independent.

### IV. Long-Term Benefits of Treatment

Short-term administration of an inhibitor of ADK activity or expression provides a long-term benefit to the subject over a benefit period (interchangeably termed a symptom-attenuation period or a recovery period). The benefit period generally overlaps the stoppage period and may begin during the treatment period or at any time after the end of the treatment period. The duration of the benefit period may, for example, be at least about 6 months, one year, 18 months, or two years, because the development and progression of epilepsy can occur on at least that time scale. Also, or alternatively, the duration of the benefit period may be at least about 5, 10, 15, 20, 25, or 50 times the duration of the treatment period.

The long-term benefit over the benefit period for a non-epileptic subject, who is at risk of developing epilepsy due to a precipitating event, is a reduced chance of developing an acquired form of epilepsy for the duration of the benefit period. The respective probabilities of the non-epileptic subject developing an acquired form of epilepsy, without and with treatment using the inhibitor, can be calculated based on statistical data (e.g., obtained in clinical trials of the inhibitor). A difference between these probabilities, where the probability is lower with the inhibitor, corresponds to a reduced chance of developing epilepsy. The chance of developing epilepsy may be reduced by any suitable amount, such as at least 10%, 25%, or 50%, among others.

The long-term benefit over the benefit period for an epileptic subject is a reduction in the chance of the subject's temporal lobe epilepsy progressing to a more severe form. The respective probabilities of the subject's epilepsy progressing to a more severe form, without and with treatment using the inhibitor, can be calculated based on statistical data (e.g., obtained in clinical trials of the inhibitor). A difference between these probabilities, where the probability is lower with the inhibitor, corresponds to a reduced chance of progression. The severity of the subject's epilepsy may be determined using any criteria, such as the frequency or average duration of seizures, an EEG, a brain MRI, and/or the like. Administration of the inhibitor may statistically reduce the frequency or average duration of seizures by at least about 10%, 25%, or 50%, among others, over the benefit period and relative to a control group of epileptic subjects.

In some embodiments, the epileptic subject may enjoy (statistically) a remission, compared to the beginning of the treatment period, and lasting at least about 3, 4, 5, 7, 10, 20, or 50 times the duration of the treatment period. The remission may be characterized by the absence of seizures, or a reduction in seizure incidence, strength, and/or average length, of at least about 10%, 25%, 50%, 75%, 90%, or 95%, among others. In some cases, the remission may be at least substantially permanent, such that the subject is deemed to be substantially free of seizures or cured of epilepsy for at least one year.

The subject may be intermittently monitored after the treatment period (e.g., during the stoppage period and/or benefit period) for at least one epilepsy-related indicator. Monitoring may or may not be performed at regular intervals. The subject may, for example, be monitored for at least about six months, one year, 18 months, or two years. The at least one epilepsy-related indicator may be a reported incidence, length, and/or severity of seizures; a characteristic electroencephalogram; a characteristic brain MRI; or the like.

### V. EXAMPLES

The following examples describe further aspects of treating subjects transiently with an ADK inhibitor to discourage epileptogenesis. These examples are for illustration only and are not intended to limit the entire scope of the present disclosure.

### Example 1

### ADK and 5-Methylcytosine (5mC) Regulation During Epileptogenesis

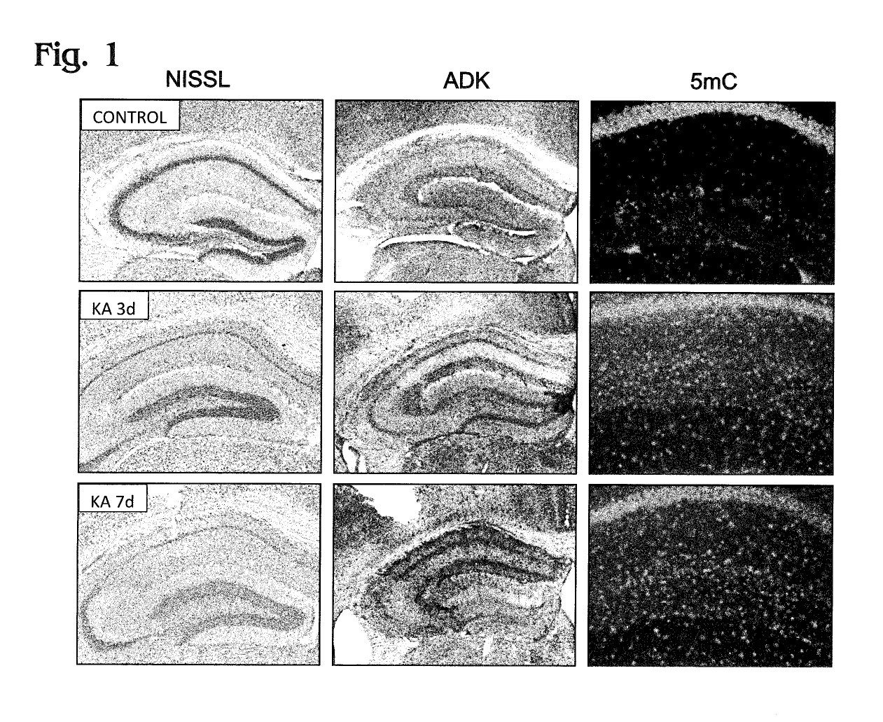

This example presents data suggesting a linkage between increased ADK levels and epigenetic modification of DNA by cytosine methylation with DNA methyl transferase (DNMT); see FIGS. 1-4.

FIG. 1 shows a set of images of mouse hippocampal sections prepared from hippocampi collected from control mice (no kainic acid (KA); top row of images) or during the latent phase of epileptogenesis in mice after intrahippocam pal injection of kainic acid to produce status epilepticus (SE). Here, SE is the precipitating event that triggers epileptogenesis. The middle row and bottom row of images respectively represent 3 days (3 d) and 7 days (7 d) after exposure to kainic acid. These time points are within the latent phase of epileptogenesis, before recurrent seizures begin. The three panels in each row are images of sections stained respectively with a Nissl stain, an anti-ADK antibody, or an anti-5-methylcytosine antibody. The images reveal an increase in astrogliosis, ADK protein, and DNA methylation in the CA1 region of the hippocampus during the latent phase after KA-triggered epileptogenesis.

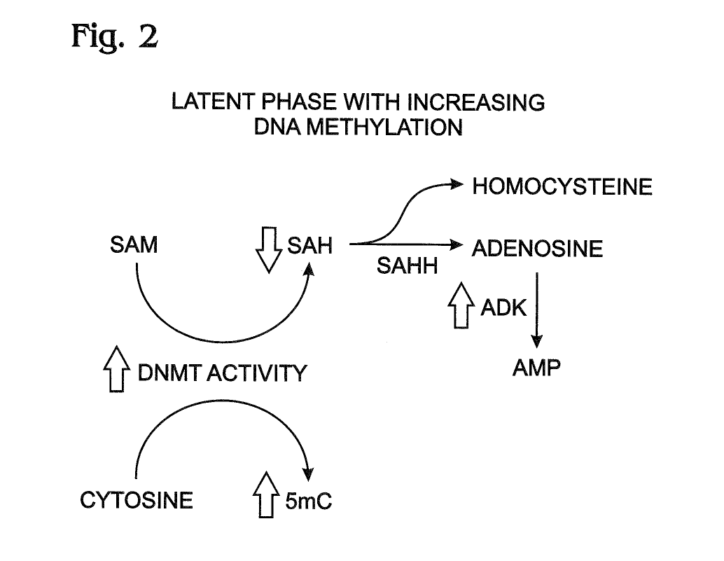

FIG. 2 shows a flowchart presenting a pair of reaction pathways that may be important during the latent phase of epileptogenesis to increase DNA methylation. The DNA methylation may function as an epigenetic modification required for epileptogenesis. The reaction pathways shown are coupled to one another by DNA methyltransferase (DNMT). DNMT catalyzes conversion of cytosine to 5-methylcytosine (5mC), while S-adenosyl methionine (SAM) acting as a co-substrate is transformed to S-adenosyl homocysteine (SAH). SAH hydrolase (SAHH) catalyzes conversion of SAH to homocysteine and adenosine, and the resulting adenosine can be modified to become adenosine monophosphate (AMP) with the help of adenosine kinase (ADK). Increasing the level of ADK during the latent phase of epileptogenesis, as indicated by an upward open arrow next to ADK in FIG. 2, reduces the level of adenosine. This reduction encourages conversion of SAH to homocysteine and adenosine, thereby decreasing the steady state level of SAH, as indicated with a downward open arrow next to SAH in FIG. 2. SAH inhibits DNMT; thus, decreasing SAH increases DNMT activity, as indicated with an upward open arrow, which increases the level of 5mC, as indicated by an upward open arrow next to 5mC in FIG. 2. In contrast, inhibiting ADK has the opposite effect, namely, decreasing cytosine methylation by DNMT, thereby preventing or eliminating epigenetic modification required for epileptogenesis.

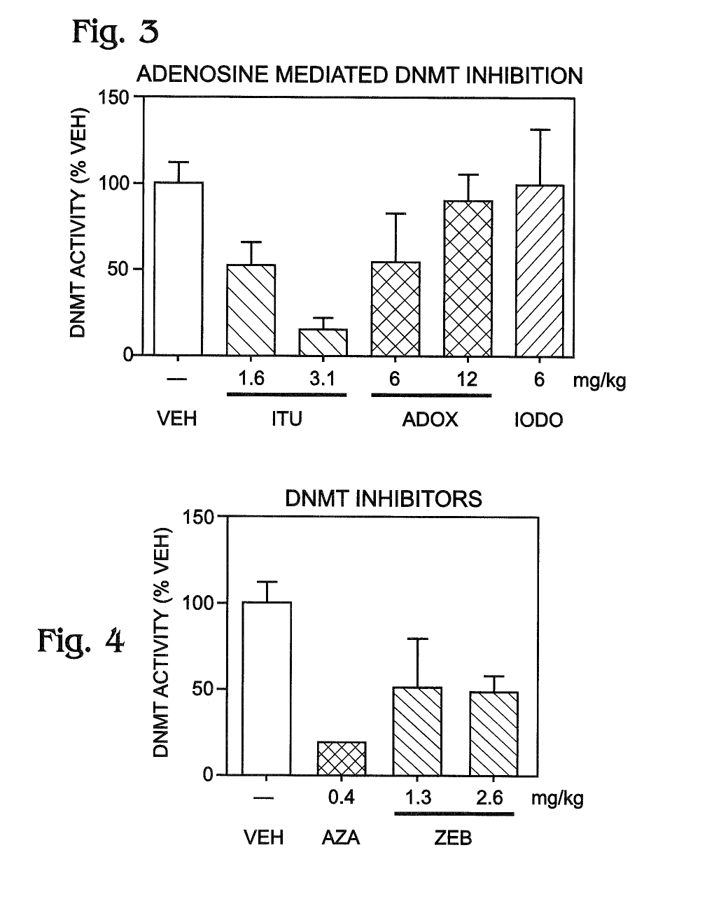

FIGS. 3 and 4 show a pair of bar graphs reporting the level of DNMT activity measured one hour after administration to mice of vehicle alone or with the indicated inhibitor. The graph of FIG. 3 shows adenosine-mediated DNMT inhibition through administration of (a) 5-iodotubercidin (ITU; an ADK inhibitor) at two different doses, (b) adenosine-2′,3′-dialdehyde (ADOX; an inhibitor of SAHH) at two different doses, and (c) 5′-iodo-5′-deoxyadenosine (IODO; another inhibitor of SAHH). ITU was found to be most effective, and reduced DNMT activity by about 50% with the lower dose and about 80% with the higher dose. The graph of FIG. 4 shows data with known DNMT inhibitors: 5-aza-cytidine (AZA) and zebularine (ZEB; a nucleoside analog of cytidine). ITU at a dosage of 3.1 mg/kg was found to be as effective as AZA for inhibition of DNMT. Therefore, treatment with ITU or other agents that inhibit ADK and/or increase adenosine levels, during the latent phase of epileptogenesis, may inhibit epigenetic modification of DNA associated with epileptogenesis. In other words, transient treatment with these agents may prevent creation of a chronic epigenetic configuration that leads to epilepsy.

### Example 2

### Transient ADK Inhibition Prevents Epileptogenesis

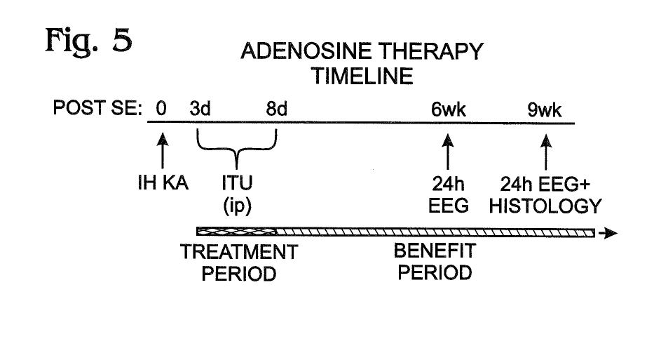

This example presents data showing the ability of transient ADK inhibition to prevent development of epilepsy in a mouse model; see FIGS. 5-14.

FIG. 5 shows a timeline for the protocol followed to trigger epileptogenesis in mice, administer an ADK inhibitor, record electrical activity (EEG), and collect tissue samples. Kainic acid (KA) was administered intrahippocampally (IH) at day 0 to produce status epilepticus. An ADK inhibitor, ITU, (or vehicle alone) was administered intraperitoneally (ip) from day 3 to day 8, for a treatment period lasting a total of five days. The ITU was injected either once per day at 3.1 mg/kg or twice per day (bid) at 1.6 mg/kg. At six weeks and nine weeks, the subjects were analyzed by EEG alone or EEG and histology, respectively.

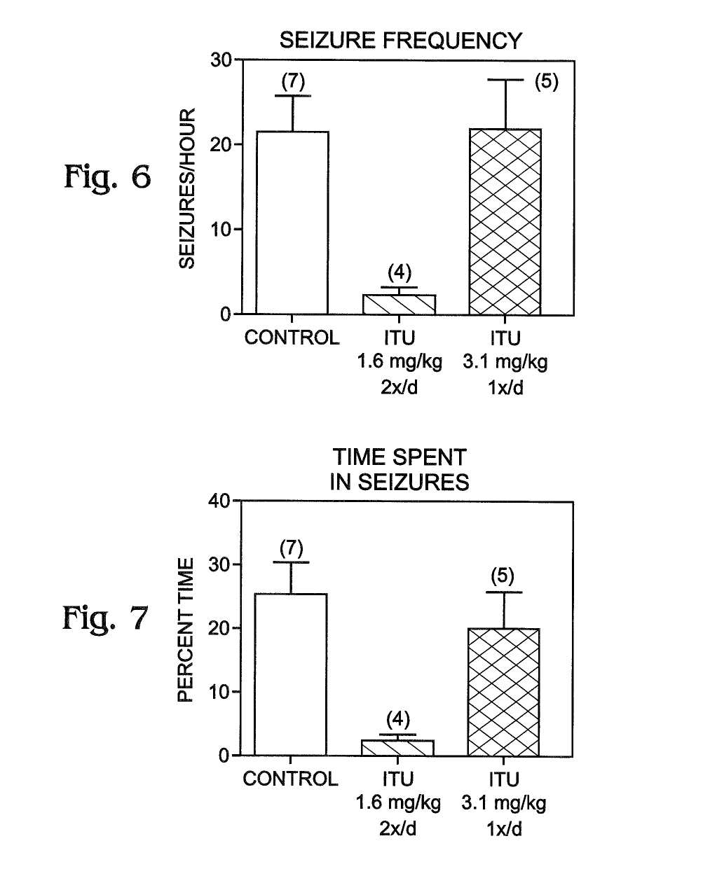

FIGS. 6 and 7 show bar graphs reporting the frequency of seizures and the time spent in seizures at six weeks after triggering epileptogenesis, as a function of ADK inhibitor (ITU) dosage, for mice treated according to the protocol of FIG. 5. The number of subjects from which data were collected is indicated in parentheses above each condition. A once-daily dose of ITU (3.1 mg/kg) was substantially ineffective, while giving half the amount of ITU (1.6 mg/kg) twice daily dramatically reduced seizure frequency and time spent in seizure. These data suggest that the half-life of ITU is much less than one day, and that ITU must be maintained above a threshold for at least a substantial portion of the treatment period to be effective at preventing epileptogenesis. The dosage frequency that is effective may be decreased with a time-release formulation or an ADK inhibitor having a longer half-life than ITU.

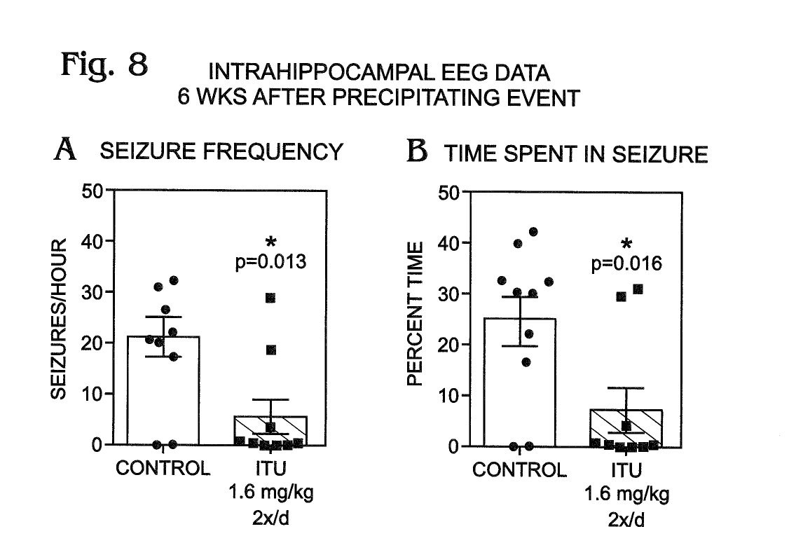

FIG. 8 shows data collected in a partial repeat of the experiment of FIGS. 6 and 7. (The ineffective once-daily dose of ITU was eliminated.) These data confirm the long-term, anti-epileptogenic effect of ITU resulting from short-term administration of ITU, as observed in FIGS. 6 and 7.

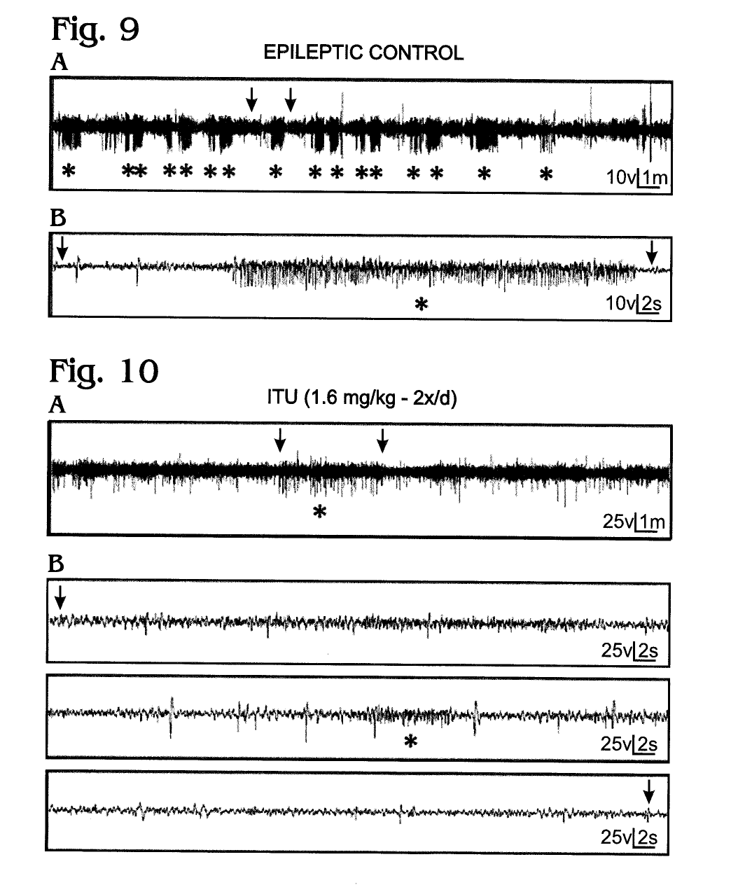

FIGS. 9 and 10 show electroencephalogram (EEG) recordings taken at the six-week time point of the protocol of FIG. 5 from a no-ITU, epileptic control subject (FIG. 9) and an ITU-treated subject (FIG. 10). The voltage scale and time scale (1 m or 2 s) are defined in the lower right hand corner for each recording. Panel A of each figure shows a 30-minute recording, while panel B of the figure shows a one-minute section (FIG. 9) or a three-minute section (FIG. 10) of the corresponding 30-minute recording. The section expanded in panel B is identified in corresponding panel A by a pair of vertical arrows. Seizures are marked with an asterisk. The individual seizure marked with an asterisk in panel B of FIG. 10 largely disappears when expanded by changing the time scale, as compared to the individual seizure marked the same way in FIG. 9.

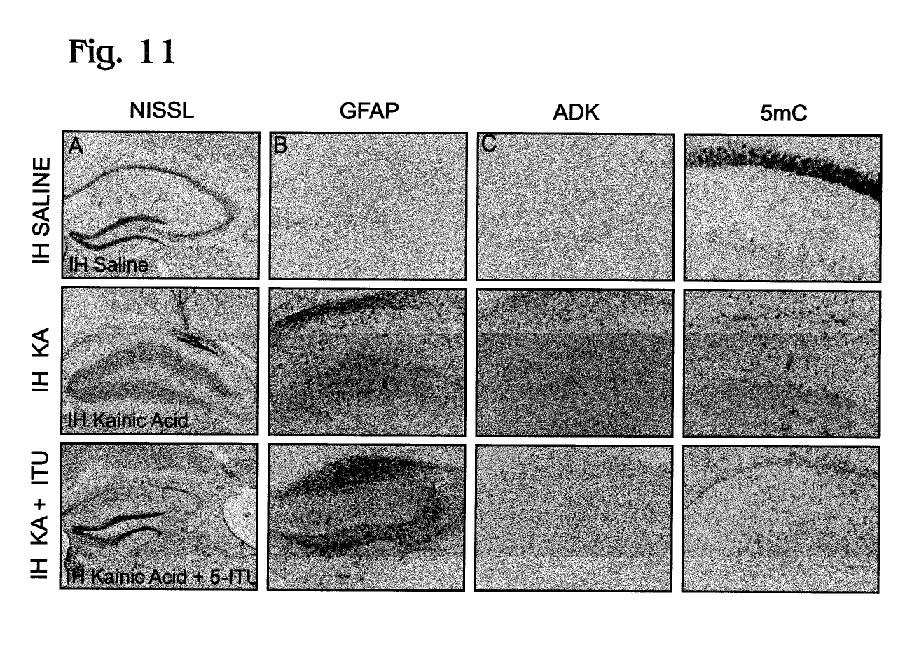

FIG. 11 shows representative images from a histological analysis of hippocampal tissue sections obtained from mice at nine weeks after producing status epilepticus by intrahippocam pal (IH) administration of kainic acid (KA). Administration of saline only is a control (top row of images). The KA-treated mice also were treated with ITU intraperitoneally (bottom row of images) according to the protocol of FIG. 5 or were treated with vehicle only (middle row of images). The tissue sections shown in the first column of images were Nissl stained, and those in the second, third, and fourth columns were stained respectively with antibodies to glial fibrillary acidic protein (GFAP), ADK, and 5-methylcytosine (5mC). Transient administration of ITU early in the protocol prevented later development of epilepsy-associated histopathology (Nissl) and reduced the levels of ADK and 5-methylcytosine observed at nine weeks.

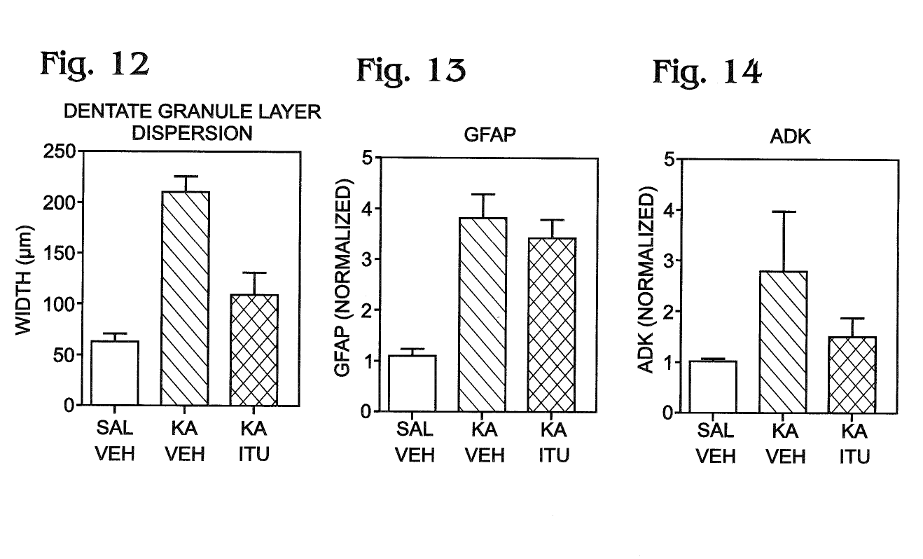

FIGS. 12-14 are a series of graphs plotting data from the histological analysis represented by FIG. 11. (SAL is saline, VEH is vehicle, KA is kainic acid, and ITU is 5-iodotubercidin.) FIG. 12 plots the width in micrometers measured from Nissl stained tissue sections resulting from the indicated treatments. FIG. 13 plots the level of GFAP, and FIG. 14 the level of ADK. The amount of dentate granule layer dispersion produced by KA is significantly less in ITU-treated mice compared to controls.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of anti-epileptogenesis treatment, the method comprising:
administering a therapeutically effective amount of an adenosine kinase inhibitor to a human non-epileptic subject over a finite, predetermined treatment period having a duration of less than two months;
wherein the non-epileptic subject has sustained a precipitating event with a known risk to trigger latent development of an acquired form of epilepsy in non-epileptic subjects,
wherein the precipitating event occurred within three months of the start of the treatment period,
wherein administration of the adenosine kinase inhibitor to the subject is stopped at the end of the treatment period for at least the longer of (i) six months and (ii) ten times the duration of the treatment period, and
wherein the step of administering reduces the chance of the subject having seizures caused by the acquired form of epilepsy for a period following the end of the treatment period and lasting at least the longer of (i) six months and (ii) ten times the duration of the treatment period.

2. The method of claim 1, wherein the duration of the treatment period is about two weeks or less.

3. The method of claim 1, wherein the precipitating event is selected from the group consisting of traumatic brain injury, hemorrhagic stroke, ischemic stroke, infection of the brain, febrile seizure, and status epilepticus.

4. The method of claim 1, wherein the adenosine kinase inhibitor is an adenosine analog.

5. The method of claim 1, wherein the adenosine kinase inhibitor is selected from the group consisting of 5-iodotubercidin, 5′-amino-5′-deoxyadenosine, ABT-702, GP-3269, and A-134974.

6. The method of claim 1, further comprising a step of intermittently monitoring the subject for an epilepsy-related indicator after the end of the treatment period, wherein the step of intermittently monitoring is conducted for at least one year.

7. The method of claim 1, wherein the step of administering includes a step of administering the ADK inhibitor orally.

8. The method of claim 1, wherein the step of administering is performed by the subject.

9. The method of claim 1, wherein the step of administering is performed by a medical practitioner.

10. The method of claim 1, the step of administering being a first step of administering, further comprising a second step of administering a therapeutically effective amount of an adenosine kinase inhibitor to the non-epileptic subject over a finite, predetermined treatment period after the first step of administering.

11. The method of claim 1, wherein the step of administering statistically results in at least a 50% chance of at least a 50% reduction in seizure incidence over a period of at least one year following the treatment period.

12. A method of anti-epileptogenesis treatment, the method comprising:
administering a therapeutically effective amount of an adenosine kinase inhibitor to a human epileptic subject over a finite, predetermined treatment period having a duration of less than two months;
wherein the epileptic subject has received a first diagnosis of temporal lobe epilepsy within one year preceding the start of the treatment period,
wherein administration of the adenosine kinase inhibitor to the subject is stopped at the end of the treatment period for at least the longer of (i) six months and (ii) ten times the duration of the treatment period, and
wherein the step of administering reduces a chance of the subject's temporal lobe epilepsy progressing to a more severe form for at least the longer of (i) six months and (ii) ten times the duration of the treatment period.

13. The method of claim 12, wherein the duration of the treatment period is about two weeks or less.

14. The method of claim 12, wherein the adenosine kinase inhibitor is an adenosine analog.

15. The method of claim 12, wherein the adenosine kinase inhibitor is selected from the group consisting of 5-iodotubercidin, 5′-amino-5′-deoxyadenosine, ABT-702, GP-3269, and A-134974.

16. The method of claim 12, further comprising a step of intermittently monitoring the epileptic subject for an epilepsy-related indicator after the end of the treatment period, wherein the step of intermittently monitoring is conducted for at least one year.

17. The method of claim 12, wherein the step of administering includes a step of administering the ADK inhibitor orally.

18. The method of claim 12, wherein the step of administering is performed by the subject.

19. The method of claim 12, wherein the step of administering is performed by a medical practitioner.

20. The method of claim 12, wherein the step of administering statistically results in at least a 50% chance of at least a 50% reduction in seizure incidence over a period of at least one year following the treatment period.
